(19) United States
(12) Patent Application Publication
(10) Pub. No.: US 2012/0142083 A1
(43) Pub. Date: Jun. 7, 2012

(54) BIOFILTER

(76) Inventor: MICHAEL F. LALLY, Lawton, IA (US)

(21) Appl. No.: 13/396,168

(22) Filed: Feb. 14, 2012

Related U.S. Application Data
(63) Continuation of application No. 11/977,624, filed on Oct. 25, 2007.
Application No. 11/977,624 is a continuation-in-part of application No. 10/794,844, filed on Mar. 5, 2004, now abandoned.

(51) Int. Cl. C12M 1/00; C12S 5/00

(52) U.S. Cl. 435/266; 435/283.1

(57) ABSTRACT

A biofilter for use in treating contaminated gases is provided with a container having a layer of rubber particulate, which serves as a filter media. One preferred source of the rubber particulate is recycled tires. The rubber particulate provides a platform for the growth and maintenance of a microbial ecosystem that substantially treats the contaminated gases emanating from the waste. A plurality of different embodiments provide relatively simple inexpensive biofiltration solutions for a wide range of applications. A method of reducing odorous pollutants from products is also described wherein the product containing the pollutant or pollutants is passed through a layer of the rubber particulate material.

### CROSS-REFERENCE TO RELATED APPLICATION

This is a continuation application of Petitioner's earlier application, Ser. No. 11/977,624, filed on Oct. 25, 2007, entitled BIOFILTER, which is a continuation-in-part application of Petitioner's earlier application, Ser. No. 10/794,844, filed Mar. 5, 2004, entitled BIOFILTER, the disclosures of which are hereby incorporated herein by reference.

### BACKGROUND OF THE INVENTION

1. Field of the Invention

The present invention relates to biofilters and more particularly to biofilters using a filter media that is primarily comprised of a rubber material to efficiently and inexpensively control the volatization of organic compounds.

2. Description of the Prior Art

At the turn of the twentieth century it was discovered that a leach field could purify liquid waste before the waste could seep into the water table. It was further discovered that the leach fields eliminated the noxious odors emanating from the liquid waste before the odors could reach the air above ground. Since that discovery, biofiltration has evolved into an important method for controlling air pollution emanating from paint shops, farms, sewage treatment plants, and various setting where industrial pollution is problematic. Most modern biofilters used in these capacities are comprised of engineered beds of soil or compost. Organic waste, whether in gaseous, liquid or solid form is disposed beneath, or adjacent to, the biofilter so that the organic gases slowly diffuse through the biofilter media. Billions of microorganisms indigenous to the soil or compost media are used to convert the organic compounds to carbon dioxide and water.

While prior art biofiltration systems have proven to be effective in the treatment of organic compounds, they suffer from a number of shortcomings. Typically, compost media has a limited life span and must be completely replaced every two to five years. During its life span, the compost media requires the adjustment of PH levels. Airflow through the compost media is also inconsistent over time, as back pressure gradually increases and air channels form throughout the compost. Compost media also tends to be hydrophobic, as it is difficult to rehydrate compost after it dries out. Accordingly, compost media typically requires the attentive care of its operator and can be quite costly over the lifetime of the treatment facility due to its maintenance, removal and replacement every few years.

Soil media has several significant advantages over compost media. However, soil media biofilters can be difficult to properly construct and may have a relatively high start up cost. Moreover, the use of soil media biofilters does not easily lend itself to a wide range of applications including uses where a plurality of smaller output filters are used within a single system, such as a municipal sewer system. Many soil media systems also suffer from compaction problems over time, causing inconsistent or wholly ineffective air flow.

The treatment and purification of wastewater is one of the more crucial areas in which biofilters can be used to benefit the public health and the ecological well-being of land and water resources. Natural byproducts of wastewater treatment include the odors and noxious gases generated by microbial activity. Many prior art methods of dealing with wastewater odor control fail to completely solve the problem. Chemical addition technologies, for example, oxidize the noxious compounds in the wastewater before they are emitted as odorous gases by feeding oxidizing chemicals to the sewage. However, nonspecific reactions occurring within the high organic load of the sewage can lead to the formation of volatile organic compounds that may be highly caustic if not toxic. Wet chemical scrubbers use large volumes of potentially dangerous chemicals and the drainage generated from such systems creates yet another form of wastewater that must be treated prior to its release into the environment. Other systems utilize only a carbon filter. However, the organic gases and odorants are only removed from the passing air until the carbon is saturated. Due to the fact that the carbon does not actually treat the passing air, it must be frequently replaced. Moreover, the effectiveness of carbon filters over time may be diminished by other environmental conditions including moisture from the passing volume of air.

Accordingly, what is needed is a novel biofilter that utilizes a filter media that is simple and inexpensive to implement but that exhibits a high level of effectiveness over a perpetual lifetime, with minimal maintenance.

### SUMMARY OF THE INVENTION

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

The biofilter of the present invention is generally provided with a container having side and bottom walls, which define an inner chamber. A layer of rubber particulate is at least partially disposed within the inner chamber of the container. In one preferred embodiment the bottom wall of the container is provided with a plurality of apertures to allow the passage of contaminated gases upwardly through the container and the rubber particulate. A cover may be provided for the container so that the biofilter could be used in place of a manhole cover for a sewer system. In one preferred embodiment the rubber particulate is provided in the form of fine ground or chip-shaped rubber, preferably derived from steel-free recycled tires. The shapes of the rubber particulate and its porous surfaces provide an ideal platform on which the naturally occurring microbial ecosystem can thrive.

In another embodiment, an open container is provided with a layer of rubber particulate, which floats on a layer of fluid within the container. Optional conduits may deliver contaminated gas, sludge and/or solid waste to the container. Contaminated gases emanating from the waste at the bottom of the container rise through the fluid and are substantially reduced by the microbial ecosystem which inhabit the rubber particulate.

It is therefore one of the principle objects of the present invention to provide a biofilter for use in treating contaminated gases using a filter media comprised substantially of rubber particulate.

It is a further object of the present invention to provide a biofilter for use in treating volatile organic compounds using a filter media comprised of rubber particulate formed from recycled products such as automobile tires.

Still another object of the present invention is to provide a biofilter system for treating contaminated gases that is adaptable for use in small scale situations including manhole covers for sewer systems.

A further object of the present invention is to provide a biofilter having a filter media that is comprised of layers of activated carbon and rubber particulate.

Still another object of the present invention is to provide a biofilter that is effective in treating contaminated gases and odors, while remaining relatively inexpensive and easy to maintain.

These and other objects of the present invention will be apparent to those skilled in the art.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 is a perspective view of one possible embodiment of a wastewater treatment facility incorporating an embodiment of the biofilter of the present invention;

FIG. 2A is a partial cut-away view of one potential source of the rubber particulate filter media of the present invention;

FIG. 2B depicts an example of chip-shaped rubber particulate as the same might be used in the filter media of the present invention;

FIG. 2C depicts an example of fine ground-shaped rubber particulate as the same might be used in the filter media of the present invention;

FIG. 3 is a partial cut-away view of one embodiment of the biofilter of the present invention as the same could be used for treating wastewater;

FIG. 4 is a cut-away view of another embodiment of the biofilter of the present invention as the same could be used in conjunction with a municipal sewer system;

FIG. 5 depicts a table that demonstrates the effectiveness of one embodiment of the biofilter of the present invention as the same could be used to control odors emanating from contaminated gas over a length of time; and

FIG. 6 depicts a table that demonstrates the effectiveness of one embodiment of the biofilter of the present invention as the same could be used to control the volatilization of contaminated gas over a length of time.

### DESCRIPTION OF THE PREFERRED EMBODIMENT

Embodiments are described more fully below with reference to the accompanying figures, which form a part hereof and show, by way of illustration, specific exemplary embodiments. These embodiments are disclosed in sufficient detail to enable those skilled in the art to practice the invention. However, embodiments may be implemented in many different forms and should not be construed as being limited to the embodiments set forth herein. The following detailed description is, therefore, not to be taken in a limiting sense in that the scope of the present invention is defined only by the appended claims.

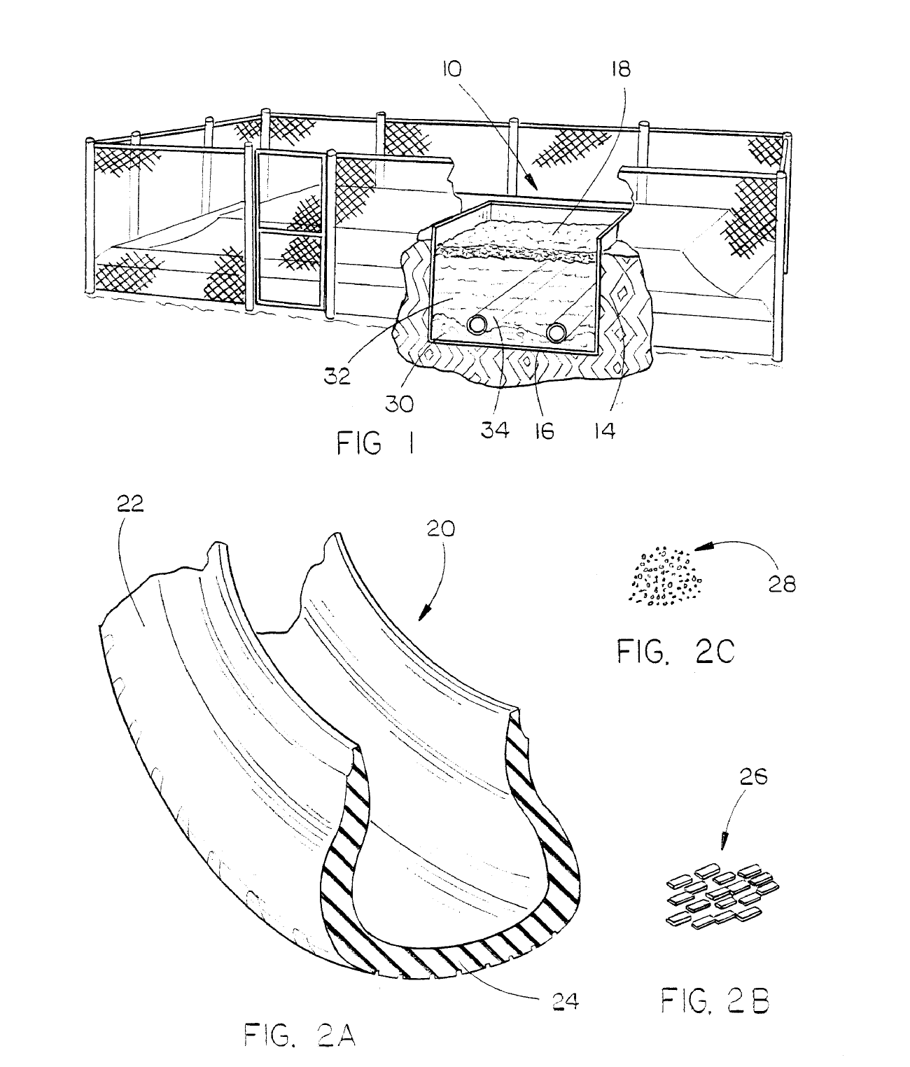

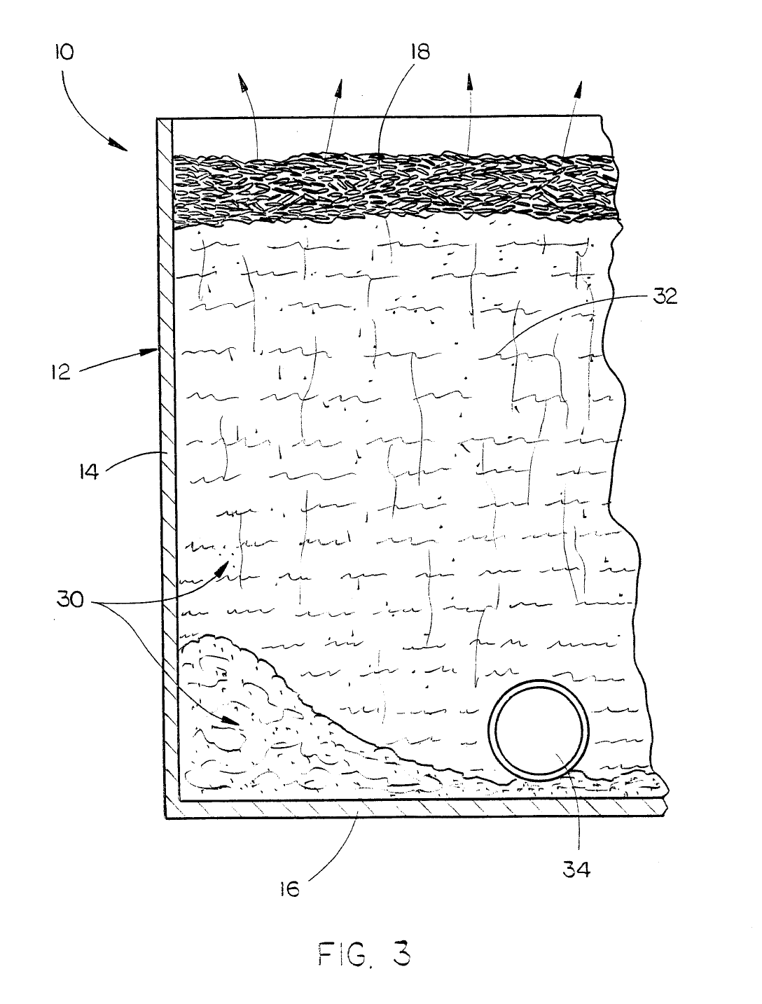

The biofilter 10 of the present invention is generally depicted in various embodiments in FIGS. 1, 3 and 4. The biofilter 10 is preferably provided with a container 12 having side walls 14 and a bottom wall 16. A layer of particulate rubber 18 is at least partially disposed within the container 12. In one preferred embodiment the rubber particulate is obtained from recycled rubber products, such as the automobile tire 20 depicted in FIG. 2A. Although it is contemplated that substantially all portions of the recycled tire 20 and other rubber based products could be used, it is preferred that those portions having steel reinforcing wires or other such foreign matter be avoided or used sparingly due to the undesirable nature of long term exposure of such materials to wet environments, which may cause the foreign matter to oxidize. However, the sidewall 22 and tread 24 of most modem passenger vehicle tires will likely be sufficiently free of such foreign matter for many of the contemplated uses for the biofilter 10.

The chipped rubber 26, depicted in FIG. 2B, and fine ground rubber 28, depicted in FIG. 2C, provide optimal shapes for use as the filter media in the biofilter 10. Both the chipped and the fine ground shapes are fairly irregular in nature, providing a large surface area for each individual piece. This, combined with the porous nature of the rubber provides an optimal platform for the formation and maintenance of a microbial ecosystem, which naturally occurs in the treatment of organic waste material. Moreover, the irregular shape of the chipped and fine ground rubber allow the particulate layer 18 to settle into a loosely packed layer that permits a consistent flow of gas through the layer of particulate rubber 18 over extended periods of time. However, the irregular shape of the particulate function to “interlock” the pieces of particulate to one another to sufficiently reduce the incidence of erosion caused by wind and weather where the layer of rubber particulate 18 is directly exposed to the elements.

After the organic material 30 passes through the layer of rubber particulate 18, the layer of rubber particulate 18 will substantially recover any openings formed by the passing organic material 30.

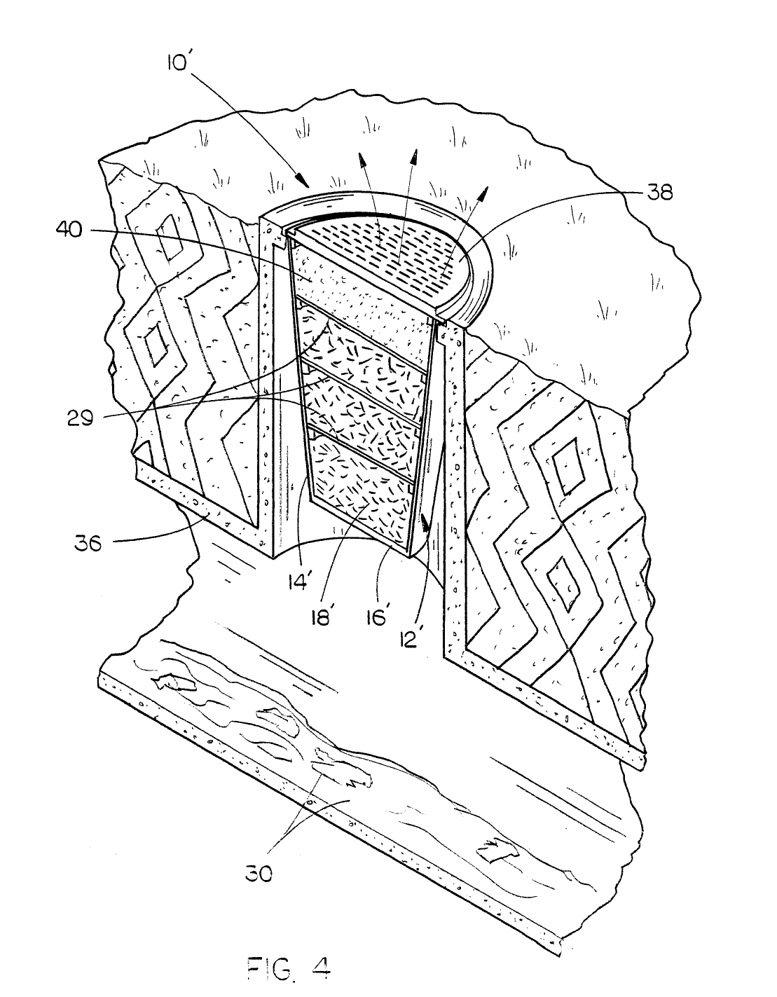

One contemplated embodiment of the biofilter 10 of the present invention is depicted in FIG. 3, which closely resembles an open-air lagoon typically utilized for liquid and/or solid organic waste 30. Depending on the particular application and the specific organic waste 30 being treated, the side and bottom walls of the container 12 could be comprised of nearly any material, such as concrete, rubber, plastic, and various non-corrosive metals. It is further contemplated that the side walls 14 and bottom wall 12 could be comprised of earthen materials, as the container could be a lagoon formed directly in the ground adjacent an organic waste producing facility. The organic waste 30 may be dumped directly into the open upper end of the container 12 since the organic waste 30, regardless of its composition, will substantially pass through the layer of rubber particulate 18 and settle at the bottom of the container 12 or become partially suspended within the layer of fluid 32. In many applications, the fluid 32 will simply be comprised of water but may be comprised of sludge or other known organic slurry. Air moving within or around the layer of rubber particulate 18 may serve as the layer of fluid where the layer of rubber particulate 18 is disposed directly on top of the organic waste 30 or a delivery means therefore. A system of conduit 34 or the like could be used as such a delivery means to deliver the organic waste 30 and/or fluid 32 to the container 12 from an adjacent or remote organic waste producing facility when top-loading of such materials is not practical or otherwise desirable.

Regardless of the manner in which the organic waste 30 is delivered to the container 12, a naturally occurring microbial ecosystem will begin breaking down the organic waste 30 within and below the layer of fluid 32. This microbial ecosystem will also inhabit the layer of rubber particulate 18 and feed on the contaminated gases delivered upwardly through the layer of fluid 32 to the layer of rubber particulate 18.

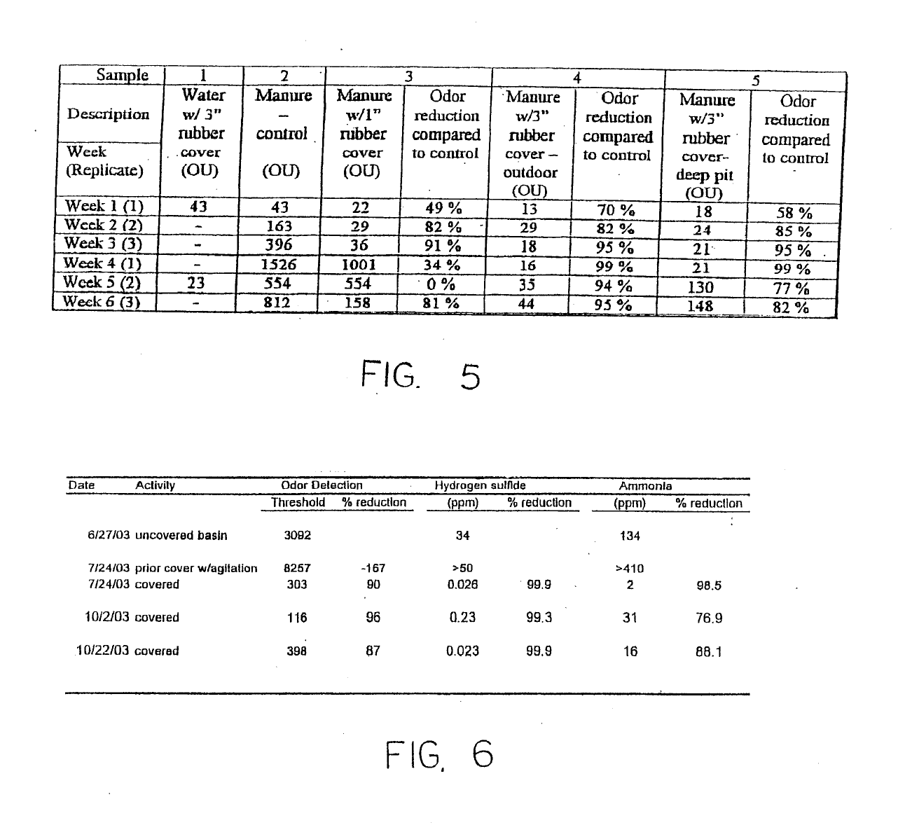

A test facility was created to quantify the benefits of the biofilter 10 as the same could be used in the treatment of organic waste within a manure slurry pit that was set up similarly to that depicted in FIG. 3. A six week testing and sampling of the manure storage containers was completed and the results are presented in FIG. 5. The contents of the manure storage tanks were similar to those typically observed in under-barn pit storage. Odor reduction was studied for one inch layer of rubber particulate (sample 3) and three inch layer of rubber particulate with reference to a control tank (sample 2). For the three inch layer, experiments were based on the mode of addition of manure to the storage structure simulating an under-barn pit (sample 5) and an outdoor storage unit (sample 4). A container filled with water and a three inch layer of rubber particulate (sample 1) was used to obtain background readings for the rubber particulate. Sludge, lagoon top water and manure for these experiments were produced from a swine facility.

As the table in FIG. 5 indicates, the one inch layer of rubber particulate resulted in more than eighty percent odor reduction during sampling weeks 2, 3, and 6. Odor reduction diminished in other weeks where high ambient temperatures were experienced or the manure additions were made by dropping the waste through the layer of rubber particulate, simulating under-barn tank conditions, thus temporarily disbursing portions of the layer of rubber particulate and exposing the waste being stored below. Performance of the three inch layer of rubber particulate was superior compared to the one inch layer of rubber particulate, effecting an odor reduction to the extent of eighty to ninety five percent, irrespective of the manner in which the manure was added to the tanks or the ambient temperature. Other important facts discovered in the testing of the layers of rubber particulate include a ninety nine percent reduction of hydrogen sulfide and a ninety eight percent reduction in ammonia, as shown in the table depicted in FIG. 6.

The biofilter 10 of the present invention is sufficiently simple in its structure and design that it is easily used as a much smaller biofilter than that depicted in FIG. 1 or 3. For example, it is contemplated that a plurality of biofilters such as the biofilter 10′ depicted in FIG. 4 could be used throughout a waste treatment system, such as a municipal sewer system. In that particular application, the container 12′ will preferably be provided with a sidewall 14′ and a bottom wall 16′. The bottom wall 16′ will preferably have one or more apertures formed therethrough that are sized and shaped to substantially prevent the passage of the layer of rubber particulate 18′ therethrough. However, the apertures within the bottom wall 16′ will permit the contaminated gases emanating from the organic waste 30, which flows beneath the biofilter 10′ within the conduit 36, to pass through to the layer of rubber particulate 18′. A slightly increased pressure of the air within the conduit 36 will tend to direct the contaminated gases upwardly through the bottom wall 16′ and through the layer of rubber particulate 18′ which will host the naturally occurring microbial ecosystem. When a cover 38 is used, such as a manhole cover, it should be provided with a plurality of apertures similar to those formed within the bottom wall 16′ so that the treated air may freely pass therethrough.

It is contemplated that the layer of rubber particulate 18′ could be divided into a plurality of layers using apertured dividing plates 39 that are coupled to the side walls 14′. Additionally, a layer of activated carbon 40 may be provided to absorb a substantial portion of the small amount of contaminated gases that may pass beyond the layer of rubber particulate 18′.

The biofilter 10′ is simply one example of the flexibility provided by the design of the biofilter of the present invention. The functionality of the biofilter 10′ will be nearly identical to that of the biofilter 10 and will be expected to have similar success in the treatment of the contaminated gases emanating from the organic waste 30.

The layer of particulate material described above in detail is also useful in reducing the content of hydrogen sulfide, ammonia, aldehydes, ketones, amines, aliphatic hydrocarbons and aromatic hydrocarbons. In such a case, the layer of particulate material removes odorous pollutants through a biological process and/or physiochemical process. In this method, the product containing the one or more hydrogen sulfide, ammonia, etc. is passed through the layer of rubber particulate material with the layer of particulate material removing a large portion of the odorous air pollutant.

The use of recycled tires in particulate form makes the filter medium easy to apply and nearly maintenance free over an indefinite lifetime. Moreover, the use of recycled materials provides an added benefit to the environment.

Any of the contemplated structural embodiments of the biofilter will be appropriate for use in the treatment of low and high volume contaminating air streams that are characterized by a low or high concentration of a plurality of different gases and compounds. The biofilter is particularly well suited for the treatment of hydrogen sulfide, ammonia, aldehydes, ketones, amines, aliphatic hydrocarbons and aromatic hydrocarbons. The use of recycled tires in particulate form makes the filter media easy to apply and nearly maintenance free over an indefinite lifetime. Moreover, the use of recycled materials provides an added benefit to the environment.

In the drawings and in the specification, there have been set forth preferred embodiments of the invention and although specific items are employed, these are used in a generic and descriptive sense only and not for purposes of limitation. Changes in the form and proportion of parts, as well as a substitution of equivalents, are contemplated as circumstances may suggest or render expedient without departing from the spirit or scope of the invention as further defined in the following claims.

Thus it can be seen that the invention accomplishes at least all of its stated objectives.

Although the invention has been described in language that is specific to certain structures and methodological steps, it is to be understood that the invention defined in the appended claims is not necessarily limited to the specific structures and/or steps described. Rather, the specific aspects and steps are described as forms of implementing the claimed invention. Since many embodiments of the invention can be practiced without departing from the spirit and scope of the invention, the invention resides in the claims hereinafter appended.

I claim:

1. The method of removing an odorous pollutant from gases emanating from an organic waste product, comprising the steps of:
providing a container having side walls, a perforated bottom wall and an open upper end which is in communication with the atmosphere;
placing a layer of particulate material in the container with the layer of particulate material consisting of rubber;
passing the gases upwardly through the perforated bottom wall of the container and through the layer of particulate material whereby a microbial ecosystem will be created within the layer of particulate material which will feed on and break down the odorous pollutant in the gases passing therethrough to remove at least some of the odorous pollutant from the gases before the gases pass through the open upper end of the container.

2. The method of claim 1 further including the step of passing a stream of the organic waste product past the perforated bottom wall of the container so that gases emanating from the stream of organic waste product will pass upwardly through the perforated bottom wall of the container.

3. The method of claim 1 wherein the pollutant is hydrogen sulfide.

4. The method of claim 1 wherein the pollutant is ammonia.

5. The method of claim 1 wherein the pollutant is an aldehyde.

6. The method of claim 1 wherein the pollutant is a ketone.

7. The method of claim 1 wherein the pollutant is an amine.

8. The method of claim 1 wherein the pollutant is an aliphatic hydrocarbon.

9. The method of claim 1 wherein the pollutant is an aromatic hydrocarbon.

10. The method of removing an odorous pollutant from gases emanating from an organic waste product contained within a container having an open upper end which is in communication with the atmosphere, comprising the steps of:
placing a horizontally disposed layer consisting of rubber particulate material over the organic waste product so that gases emanating from the organic waste product will pass upwardly through the layer of rubber particulate material whereby a microbial ecosystem will be created within the layer of rubber particulate material which will feed and break down the odorous pollutant in the gases passing upwardly through the layer of rubber particulate material to remove the odorous pollutant from the gases before the gases reach the atmosphere.

11. The method of claim 10 wherein the pollutant is hydrogen sulfide.

12. The method of claim 10 wherein the pollutant is ammonia.

13. The method of claim 10 wherein the pollutant is an aldehyde.

14. The method of claim 10 wherein the pollutant is a ketone.

15. The method of claim 10 wherein the pollutant is an amine.

16. The method of claim 10 wherein the pollutant is an aliphatic hydrocarbon.

17. The method of claim 10 wherein the pollutant is an aromatic hydrocarbon.

18. A biofilter for removing odorous pollutants from an organic waste product, comprising:
a container having an organic waste product therein;
a layer of particulate material consisting of rubber in said container which is positioned above or below the organic waste product so that gases emanating from the organic waste product will pass through the layer of particulate material whereby a microbial ecosystem will be created within the layer of particulate material which will feed on and break down the odorous pollutant in the gases passing therethrough to remove at least some of the odorous pollutant from the gases before the gases pass from said container.

19. The biofilter of claim 18 wherein the rubber is fine ground particles having an irregular shape.

20. The biofilter of claim 18 wherein the rubber is chip-shaped with the chips having a generally flat rectangular configuration.

21. The biofilter of claim 18 wherein the organic solid waste product is a liquid or solid organic waste product.
